(19) United States
(12) Patent Application Publication
(10) Pub. No.: US 2025/0075240 A1
(43) Pub. Date: Mar. 6, 2025

(54) COMPOSITIONS & METHODS FOR ARCHITECT OLIGO MEDIATED DNA SYNTHESIS

(71) Applicant: Duke University, Durham, NC (US)

(72) Inventors: Eirik Adim Moreb, Durham, NC (US); Michael David Lynch, Durham, NC (US)

(21) Appl. No.: 18/726,436

(22) PCT Filed: Jan. 5, 2023
(86) PCT No.: PCT/US23/60131

§ 371 Date: Jul. 3, 2024

Related U.S. Application Data
(60) Provisional application No. 63/266,413, filed on Jan. 5, 2022.

(51) Int. Cl. C12P 19/34

(52) U.S. Cl. CPC C12P 19/34

(57) ABSTRACT

A DNA synthesis technology that relies on sequence-directed, multiplexed ligations to enable template-independent, exponential synthesis of gene- or genome-length DNA. This approach relies on well characterized and optimized enzymes and thus does not require further protein engineering. This approach is amenable to cost-effective automation and thus will enable cost-effective DNA “printers”.

### CROSS-REFERENCE TO RELATED APPLICATIONS

This application claims benefit of U.S. Provisional Application No. 63/266,413, filed Jan. 5, 2022, which application is incorporated herein by reference in its entirety.

### FIELD OF INVENTION

This invention relates to methods of DNA synthesis. Using a limited set of “architect oligos”, DNA synthesis is accomplished by iterative rounds of oligonucleotide directed extension, ligation and cleavage. These cycles can be multiplexed for efficient synthesis

### REFERENCE TO A SEQUENCE LISTING

The instant application contains a Sequence Listing which has been filed electronically in ASCII format as 47381-69.xml created on Jan. 4, 2023 and is 39026 bytes in size and is hereby incorporated by reference in its entirety.

### BACKGROUND

According to BCC Research, the current synthetic biology market will soon exceed $18 Billion USD annually. This growth is in large part driven by key advances in technologies to both read and write DNA. The market for DNA or gene synthesis products alone is expected to exceed $7 Billion USD by 2024. The cost of synthesis has lagged significantly behind the reductions seen in the cost of DNA sequencing and on a per base pair level synthesis is still 5 orders of magnitude higher than that of DNA sequencing. At current best prices for DNA synthesis (of ˜$0.05-$0.15/bp) the synthesis of a relatively simple bacterial genomes, such as E. coli (˜5 Mbp) would still cost ˜$350,000, which is intractable for routine experimentation. Additionally, the lowest reported costs per base pair are often not realized in practice. From recent purchases, the cost of a 4 kbp “gene” ranges anywhere $675.00 (˜$0.14/bp) for a sequence verified clone to $575.00 for linear DNA fragments which need to be assembled and cloned. This corresponds to over $0.16/bp. In addition to the fact that the actual costs for longer sequences are higher than the lowest price points, many additional “difficult” to manufacture sequences cannot be obtained from DNA synthesis providers. For example GC or AT rich sequences as well as sequences with repetitive elements need to be cloned with more traditional methodology. For the field of synthetic biology to realize its true potential, the cost of writing DNA needs to be reduced by 100- to 1000-fold to make routine DNA synthesis (of even large or difficult sequences) a feasible tool for routine systematic experimentation even in academic labs. Ideally, to be game changing, DNA synthesis technologies should be as simple and as affordable as PCR.

### SUMMARY

This invention is a next generation DNA synthesis technology. The process, illustrated in FIG. 1, has the potential to overcome many of the challenges associated with current methods of DNA synthesis and as a result also has the potential to enable extremely low costs for DNA synthesis and assembly. Traditional methodologies all still rely on the synthesis of oligonucleotides and the use of DNAs double stranded nature and enzymes to build larger dsDNA fragments. While the cost of oligonucleotide synthesis has dropped significantly, a key limitation is the ability to assemble oligonucleotides into larger genes. Assembly has mostly utilized double-stranded DNA ligases, polymerase cycling assembly (PCA), or variations therein. These methods are limited by the inherent error rates in oligo synthesis (coupling efficiencies) and high complexity present in multiplexed-gene synthesis (providing an upper limit to the cost savings of oligo pool gene assemblies). Thus the cost of larger DNA fragments hasn't decreased with the drop in cost of oligonucleotide synthesis and sequencing. This approach overcomes many of these challenges and enables template independent, exponential DNA synthesis, for example 2 bp to 4 bp to 8 bp to 16 bp, . . . etc., One non-limiting example of this process is illustrated in FIG. 1. Exponential growth enables the potential to synthesize DNA fragments of up to 10 kilobases in less than 14 cycles (which theoretically, can be achieved in an overnight “PCR type” reaction and is less cycles than a 15 bp oligo), reducing cycle number and compounding errors associated with oligo building technologies.

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The invention includes Architect oligonucleotides and Donor oligonucleotides.

Each Architect oligonucleotide comprising: an anchor sequence operably linked to an architect sequence. Donors oligonucleotide comprising: an architect complimentary sequence operably linked to a cleavage site that is operatively linked to a subsequence of an oligonucleotide product to be synthesized.

In some aspects, the invention encompasses a template independent, exponential method of synthesizing an oligonucleotide product. Firstly a pair of Architect oligonucleotides and Donor oligonucleotides are provided and mixed together. The method then conducts on the Architect oligonucleotides and Donors oligonucleotides mixture repeated cycles of Extend, Ligation, Amplification, and Cleavage. Each cycle enlarges the subsequence of the oligonucleotide product until the subsequence represents the complete oligonucleotide product.

Other methods, features and/or advantages is, or will become, apparent upon examination of the following FIGs and detailed description. It is intended that all such additional methods, features, and advantages be included within this description and are protected by the accompanying claims.

### BRIEF DESCRIPTION OF DRAWINGS

The novel features of the invention are set forth with particularity in the claims. A better understanding of the features and advantages of the present invention will be obtained by reference to the following detailed description that sets forth illustrative aspects, in which the principles of the invention are used, and the accompanying drawings of which:

FIG. 1A-1F: Architect oligos are used to direct Donor oligo ligations and improve efficiency. 1A) To ligate i) donor sequence 1 to ii) donor sequence 2, four oligos are used. The Donor oligos each include: donor sequence (i) & (ii), cut sites (iii) & (iv), and Architect binding sequences (v) & (vi) and have a 5′ phosphate group. The Architect oligos each include: Architect binding sequences (v) & (vi) and are operably linked to a (vii) the Anchor sequence. In FIG. 1B) The Anchor sequence connects the Architect oligos via overlapping 5′ ends. The Donor oligos then bind the Architect oligos in a sequence-specific manner. A polymerase is used to Extend the Architect oligos, filling in the 5′ overhang created by the Donor oligos binding to the Architect oligos. This creates dsDNA ends which can then be ligated together in what is effectively an intramolecular ligation (highlighted in the dashed circle). FIG. 1C) To evaluate this method, qPCR primers were designed to bind to the Architect sequences. FIG. 1D) Lower Cq values show increased concentrations of the target sequence compared to negative controls in which either the polymerase was not included (demonstrating the requirement to use dsDNA) or the Architect oligos were not included (demonstrating that the Architect oligos are required for improved efficiencies of intramolecular reactions vs intermolecular reactions as well as being used to direct the ligations). FIG. 1E) To confirm the ligations were working as expected, the target sequence was amplified, cloned into a pSMART backbone using Golden Gate Assembly, and colonies were isolated and sequenced. FIG. 1F) Sequencing results confirmed Donor sequence 1 and Donor sequence 2 were ligated together (SEQ ID NO: 15).

FIG. 2A-2H: Architect oligos enable multiplexed, directed ligations of Donor oligos. 2A) Architect oligos can be used to ligate i) Donor sequence 1 to ii) Donor sequence 2 and viii) Donor sequence 3 to ix) Donor sequence 4 in a single reaction. Donor oligos have a 5′ phosphate group at the end of the Donor sequence and contain a 3′ sequence that is complementary to a specific Architect sequence. In this case, v) Architect 1 and vi) Architect 2 are used to direct the ligation of Donor 1 and Donor 2 while x) Architect 3 and xi) Architect 4 direct the ligation of Donor 3 and Donor 4. Both Donor 1 and Donor 3 oligos contain a iii) Cut Site 1 between the Donor and Architect sequences. Both Donor 2 and Donor 4 oligos contain a iv) Cut Site 2 between the Donor and Architect sequences. Architect oligos all have the vii) Anchor sequence at their 5′ end such that Architect 1 and 3 can bind to either Architect 2 or 4 but not to each other. 2B) All four Donor oligos are mixed with all four Architect oligos. i) The oligos anneal to each other and ii) the 3′ overhangs are filled in by a polymerase to create dsDNA. iii) A ligase is then used to join the ends of the Donor oligos such that the sequence Donor 1 is joined to the sequence Donor 2 and the sequence Donor 3 is joined to the sequence Donor 4. 2C) Primers binding to the Architect sequences can then be used to specifically amplify these sequences, which can be assayed using qPCR. 2D) As a control for qPCR, the assay was repeated but excluded Architect 4. 2E) This allows specific amplification from qPCR of Architect 1 and 2 but not from Architect 3 and 4. 2F) The Cq values from the qPCR show amplification from earlier cycles when amplifying Architect 1 and 2 from the condition with all four Architects and from the control condition. However, amplification from Architect 3 and 4 shows a lower Cq value only in the presence of the fourth Architect. This was further confirmed using Golden Gate Assembly to clone the amplified products, isolate colonies, and sequence the cloned product. Shown is a chromatogram for 2G) Donor sequence 1 joined to Donor sequence 2 (SEQ ID NO: 16) and 2H) Donor sequence 3 joined to Donor sequence 4 (SEQ ID NO: 17).

FIG. 3A-3D is Selective digestion of PCR amplified Architect-directed ligation products enables multiple cycles of directed ligations. 3A) All four Donor oligos are mixed with all four Architect oligos as described. In FIG. 2. After an Extend and a Ligate step, sequences Donor 1 and Donor 2 are joined and sequences Donor 3 and Donor 4 are joined. 3B) PCR amplification from these ligated products using primers that bind to the Architect sequences results in linear Donor DNA flanked by Architects. Cleavage Site 1 and 2 enable selective cleavage of either Architect sequence. 3C) When mixed with new Architect 1 and Architect 4 oligos, this enables an Extend and Ligate step to result in sequences Donor 1-Donor 2 to be joined with sequences Donor 3-Donor 4, such that the sequence order is Donor 1-Donor 2-Donor 3-Donor 4. 3D) When the Donor are cleaved by Type IIS enzymes, it leaves a 5′ overhang but only the strand with the overhang is used in the subsequent Architect-directed ligation. 3E) To confirm this, we cloned the product of c) using Golden Gate Assembly, isolated clones and sequenced the results. Shown is sequence confirmation of Donor 1-Donor 2-Donor 3-Donor 4 ligated together (SEQ ID NO: 18).

FIG. 4A-4E Using a standardized set of Donor oligonucleotides, Architect-directed ligations can be used to synthesize any sequence of DNA. 4A) In this example, Donor oligonucleotides consist of a 3′ sequence that is complementary and can bind to an Architect sequence (shown here in (Architect 1), (Architect 3), (Architect 2), or (Architect 4), a Cut Site that is cleavable by a Type IIS restriction enzyme, and a 5′ single nucleotide (“Donor” DNA) which becomes part of the synthesized DNA. Required but not shown are 5′ phosphate groups. With these 16 Donor oligonucleotides, in combination with 4B) four Architect pairs, 4C) primers that bind to the Architect sequences and 4D) the following enzymes: polymerase, ligase, Type IIS restriction enzyme 1 (cleaves the 1 Cut Site), and Type IIS restriction enzyme 2 (cleaves the 2 Cut Site), any sequence can be synthesized. 4E) To make the sequence 5′-TTGA-3′, 4E (i) Donor oligonucleotides with the desired Donor DNA are selected that 4E (ii) bind to two Architect pairs. These are mixed in a single reaction in which a polymerase is first used to extend the double strand DNA from the Architect oligonucleotides and then a ligase joins the 5′ phosphorylated Donor DNA to create 4E (iii) two ligated sequences 5′-TT-3′ and 5′-GA-3′. 4E iv) The reaction is then split and PCR is used to amplify linear products containing the Donor DNA, both Cut Sites, and the Architect sequences. 4E v) The products are then cleaved using either Restriction Enzyme 1 or Restriction Enzyme 2 and the products are 4E (vii) mixed with a new Architect pair (Architect 1 and Architect 4). 4E vii) In the presence of a polymerase the DNA is denatured and annealed twice and the product is ligated together to join the Donor DNA, creating the sequence 5′-TTGA-3′. 4E viii) This product can then be used as Donor DNA for steps 4E ii) to vii), resulting in a 16 nucleotide sequence.

FIG. 5A-5H: Pairs of Architect sequences (e.g. Architect 1 (v) and 2 (vi)) can be connected in different ways. 5A) As shown in Examples 1-3, the complementary DNA of the Anchor sequence is used to link the two Architect sequences, 5B) enabling directed ligations of donor pairs. 5C) Alternatively, one or more interstrand cross-links could be introduced, 5D) enabling directed ligations without allowing the Architect sequences to denature. 5E) Alternatively, the Anchor sequence could be replaced with 5′-5′ linked Architect sequences, 5F) which would enable directed ligations without any dsDNA. 5G) Another way to attach oligos would be through a 5′ modified Architect 1 attached to an internally modified Architect 2. The link could be made with Click chemistry, for example. 5H) This would enable directed ligations along with the addition of a 5′ functional group (e.g., biotin) on the Architect sequence

FIG. 6A-6E: Uracil can be incorporated into the Architect oligonucleotides to facilitate specific cleavage and release of newly ligated DNA via USER or a combination of UDG and EndoVIII. 6A) Two Architect pairs (e.g. Architect 1 and Architect 2) are each covalently linked and have uracil (U) at one termini. 6B) Donor DNA bind to the Architect sequences and undergo Extend and Ligation steps to join the Donor DNA sequences. 6C) USER enzyme (or alternatively uracil DNA glycosylase (UDG) and Endonuclease VIII) is used to excise the uracil, leaving 5′ and 3′ phosphates. 6D) The DNA is denatured (for example be using alkaline conditions or heat) and the biotin-tagged DNA is captured magnetically via streptavidin magnetic beads. 6E) The remaining strands of DNA are used as Donor oligonucleotides in the next Architect-directed ligation

FIG. 7A-7C Asymmetric PCR using a single primer can be used to generate single stranded Donor oligos for the next cycle. 7A) Primers with a 5′ tail containing a sequence that will bind to either Cut Site 1 or Cut Site 2 can be used in an asymmetric PCR reaction to amplify from the Architect sequences such that the amplified product is single-stranded DNA containing a hairpin at the 5′ end. 7B) This hairpin then provides a substrate for sequence-specific endonuclease cleavage such that the hairpin is cleaved, leaving the Donor sequence at the 5′ end of the single-stranded oligo. 7C) This donor sequence can then be mixed with new Architect sequences to direct the next ligation, creating the next desired product.

FIG. 8A-8D: Denaturing and isolating single stranded DNA can be used to generate Donors for moving from one round of ligations to the next. 8A) A pair of Architect sequences direct the ligation of two Donor sequences (black and grey) with 5′ phosphates. A polymerase is used to fill in the 5′ overhangs created from the Donors binding to the Architects and then the Donor sequences are ligated together. This is followed by cleavage of Cut Site 1 or Cut Site 2. In this example, Cut Site 2 is cleaved. 8B) The DNA is then mixed with streptavidin magnetic beads and denatured in the presence of a magnet. Denaturation can either be temperature controlled or alkaline. Once denatured, the DNA strand with the biotin is captured by the magnet while the other strands can be moved to the next reaction. 8C) Simultaneously, another pair of Architects and Donors undergo the same reactions, except that this one is cleaved at Cut Site 1. 8D) Single-stranded Donor is then mixed with a new Architect pair to prepare for the next round of ligations.

FIG. 9A single oligo containing two Architect sequences can direct single-stranded DNA ligations and improve ligation efficiency. Cleavage and cycling of single-stranded.

FIG. 10A-10B: Architects can enable multiple cycles of directed ligations (“Architect cycles”). 10A) In the first round of an Architect cycle, the necessary steps for creating ligated Donor sequences (dashed lines) are followed: Extend, Ligation, Amplification, and Cleavage. This can result in Donor sequences that can bind to either Architect 1 and 4 or to Architect 2 and 3. The second round then introduces the appropriate Architect pair to direct the next ligation. From the second round, one of two Architect-binding Donor DNA are possible. From the reaction in which Architect 1 and 4 are used (top), Donor with sequences that bind to Architect 1 or Architect 4 can be generated by changing which Cut Site (1 or 2) is cleaved. Similarly, from the reaction in which Architect 2 and Architect 3 are used (bottom), Donor with sequences that bind to Architect 2 or 3 can be generated. Therefore, it is possible that after four unique Architect cycles, a new Architect cycle could be started using the resulting Donor sequences. 10B) The number of unique Architect sequences can also be increased. As shown, this would increase the number of Donors ligated per Architect Cycle as well as the number of rounds of ligations per Architect Cycle.

FIG. 11A-11C: The final synthesized DNA can be amplified and provided as linear or circular DNA, as desired. Primers for amplification can either 11A) bind to the Architect sequences or 11B) use custom primers that bind to the newly synthesized DNA. When using the Architect binding primers, a subsequent cleavage step would be required to remove the Architect and Cut Site sequences. Overhangs could be filled in by polymerase. 11C) When circular DNA is required, a ligase could be used to join the ends of the linear DNA. This includes instances where the synthesized DNA encodes a whole plasmid sequence.

FIG. 12: Architect oligos can be used to direct ligations between synthetic DNA and one or more existing sequences of interest. In this example, PCR is used to amplify the sequence of interest using primers that include Cut Site 2 and an Architect sequence. This can then be combined with synthetic DNA using Architect-directed ligations.

FIG. 13A-13C: Architect-directed ligations can incorporate mixed sequences as Donor DNA in order to achieve mutant libraries. 13A) A mixture of four nucleotide Donors is shown wherein the third nucleotide of the Donor (indicated by the star) is one of each possible nucleotide and the first, second and fourth nucleotides are constant. 13B) This mixture of Donor oligos is used in Architect-directed ligations in order to make 13C) a mutant library with all possible Donors incorporated

FIG. 14A-14B: Donor oligos can partially synthesized prior to use in Architect directed ligations. 14A) Part of a Donor oligo is synthesized which contains Donor Sequence 1 and Cut Site 1 and includes a 5′ phosphate group. Another oligo attached to solid supports (or other purification method) by its 5′ terminus (black horizontal line) and encodes the complementary sequence of Cut Site 1 and Architect 1 or other Architects. Additionally, a 3′ blocking group prevents strand extension in the presence of a polymerase. 14B) The overlapping complementary sequence on both oligos (Cut Site 1) allows the Donor oligo to act as a primer when binding the other oligo. In the presence of a polymerase, the 3′ end of the partial Donor oligo is extended such that it now contains the sequence of which Architect is encoded (in this example, Architect 1 is used). Subsequently, the DNA is denatured (either by heat or alkaline conditions) and the supernatant, containing the now complete Donor oligo, can be moved on to use in Architect-directed ligations.

FIG. 15A-15D: Uncleaved products can be captured and removed prior to subsequent cycles to improve cycle efficiency. 15A) We tested single cycle efficiency by starting with PCR products that are similar to the product of the amplification step and completing each step in the cycle, followed by analyzing the product of the final amplification step using NGS. 15B) We compared this to a similar workflow where we attached biotin to one of the amplified product's Architect sequences but not the other, which allowed us to use streptavidin beads to filter out unwanted byproducts following the Cleave step and before the Anneal step. 15C) Without the biotinylated DNA, a majority of the sequences included ligations of uncleaved sequences due to inefficient cleavage of Type IIS enzymes (data shown from one replicate). 15D) By including biotin-based filtering, we were able to improve average efficiency from 32.3% to 84.3%.

FIG. 16A-16D: Architect oligos can be covalently linked at their 5′ ends instead of using overlapping sequence. 16A) In Cycle 1, (i) the Architect oligo encodes two unique sequences that are linked by their 5′ ends while the (ii) Donor oligos encode sequences that are complementary to the Architect sequences, a cut site for either Esp3i or SapI (not shown), and 30 bp of “donated” DNA that becomes part of the final sequence. After annealing, (iii) a polymerase is used to “Extend” the ends of the Architect sequence, copying the “donated” DNA and making double-stranded DNA (dsDNA). (iv) These blunt ends are ligated and then (v) Amplified. The Amplified product is sent for NGS and also moved to the second cycle. 16B) In Cycle 2, the starting material is (i) Cycle 1 products from two Cycle 1 reactions. (ii) These are cleaved by either Esp3i or SapI and then (iii) annealed to a new Architect oligo through a process of denaturing and annealing. (iv) Similar to Cycle 1, a polymerase is used to “Extend” the Architect, creating dsDNA, then Ligated and Amplified. The Cycle 2 product is sent for NGS. 16C) An overview of this experimental system shows that over two cycles, 4 donor oligos are combined to make a single sequence. 16D) One of the two Cycle 1 products was sequenced using Next Generation Sequencing and resulted in 68.4+/−1.6% (n=3) of sequences being correct. In Cycle 2, 58.7+/−8.4% (n=3) of sequences were correct.

FIG. 17: A Table depicting oligonucleotide sequences used in the methods described herein.

### DETAILED DESCRIPTION

We now describe compositions and methods for template independent, exponential DNA synthesis.

### I. Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

The recitations of numerical ranges by endpoints include all numbers subsumed within that range (e.g., 0.5 to 5 includes 1, 1.5, 2, 2.75, 3, 3.80, 4, 5, etc.).

The term “about,” when referring to a value or to an amount of mass, weight, time, volume, concentration, or percentage is meant to encompass variations of ±10% from the specified amount. The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression. The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, chromosomal and native and endogenous refer to genetic material of the host microorganism.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

The terms partially and completely complementary and partially and completely hybridize or hybrid are used to describe the interaction between any oligonucleotides, polynucleotides, subsequence, or nucleic acid fragments of any length that are at least partially complimentary. The purpose of providing complementary sequences is to obtain a double stranded sequence recognizable by an endonuclease. That is to say that the hybridization between two complementary sequences needs to be sufficient to form an endonuclease recognition site but may not need to be completely perfectly hybridized or complementary to each other. There may be gaps or partially single stranded segments within a double stranded recognition sequence, yet not impede binding and cleavage by an endonuclease.

Any contiguous nucleotide sequence of a target polynucleotide is generally formed of nucleotides from the group consisting of: A, G, T, or C. Likewise, the donor and acceptor oligonucleotides are also generally formed of nucleotides A, G, T, or C. It is appreciated though that variants or structural equivalents or mimics or non-natural nucleotides may also be used in the oligonucleotides of the invention and in the target polynucleotide that is synthesized by the methods described. For example, uracil, inosine, isoguanine, xanthine (5-(2,2 diamino pyrimidine), 8-azaguanine, 5 or 6-azauridine, 6-azacytidine, 4-hydroxypyrazolopyrimidine, allopurinol, arabinosyl cytosine, azathioprine, aminoallyl nucleotide, 5-bromouracil, any isomer of any natural or non-natural nucleotide, thiouridine, queuosine, wyosine, methyl-substituted phenyl analogs, purine or pyrimide mimics may be used.

When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, DCW means dry cell weight, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “kDa” means kilodaltons, “g” means the gravitation constant, “bp” means base pair(s), “kbp” means kilobase pair(s), “% w/v” means weight/volume percent, “% v/v” means volume/volume percent, “rpm” means revolutions per minute, “HPLC” means high performance liquid chromatography, “GC” means gas chromatography, and “oligo” refers to an oligonucleotide comprising a series of contiguous nucleotides of any length.

### Overview of Invention Aspects

The invention includes in one aspect, a pair of Architect oligonucleotides that are partially complimentary to each other. Each Architect oligonucleotide comprising: an anchor sequence operably linked to an architect sequence. In one aspect, the invention includes at least one Donors oligonucleotide comprising: an architect complimentary sequence operably linked to a cleavage site that is operatively linked to a donor sequence 5′ overhang of at least 1 nt. The invention also includes a mixture of Architect oligonucleotides and Donors oligonucleotides.

In one aspect, an Architect or Donor oligonucleotides may have a purification tag. In one aspect, the pair of Architect oligonucleotides are operably linked to each other covalently.

In one aspect, any Architect oligonucleotide may include modifications rendering the oligonucleotide resistant to endonuclease cleavage.

In one aspect, an Architect oligonucleotides and Donors oligonucleotides mixture may include two or more donor oligonucleotides.

In one aspect, an Architect oligonucleotides and Donors oligonucleotides mixture may include a DNA polymerase. The DNA polymerase of the mixture may lack a 5′-3′ nuclease activity or lack strand displacement activity, or may be thermostable.

In one aspect, an Architect oligonucleotides and Donors oligonucleotides mixture may include a ligase. In some aspects, the ligase may be thermostable

In one aspect, an Architect oligonucleotides and Donors oligonucleotides mixture may include an endonuclease. In some aspects, the endonuclease may be thermostable, or may be a CRISPR or type IIS endonuclease. In some aspects the Architect oligonucleotides and Donors oligonucleotides mixture may include a UDG enzyme.

In some aspects, the invention encompasses a template independent, exponential method of synthesizing an oligonucleotide product. Firstly a pair of Architect oligonucleotides and Donor oligonucleotides are provided and mixed together. The Architect oligonucleotides are partially complimentary to each other and each Architect oligonucleotide includes an anchor sequence operably linked to an architect sequence. Further, the panel of Donors oligonucleotides, each Donor oligonucleotide characterized by: an architect complimentary sequence operably linked to a cleavage site that is operatively linked to a subsequence of the oligonucleotide product. The method then conducts on the Architect oligonucleotides and Donors oligonucleotides mixture repeated cycles of Extend, Ligation, Amplification, and Cleavage. Each cycle enlarges the subsequence of the oligonucleotide product until the subsequence represents the complete oligonucleotide product. In some aspects the method is conducted with more than one pair of Architect oligonucleotides. In some aspects the Extend includes addition to the Architect oligonucleotides and Donors oligonucleotides mixture of a polymerase. In some aspects, the Ligation includes addition to the Architect oligonucleotides and Donors oligonucleotides mixture of a ligase. In some aspects, Amplification comprises addition to the Architect oligonucleotides and Donors oligonucleotides mixture of architect complimentary primers and application of PCT suitable conditions to the mixture. In one aspect, Cleavage comprises addition to the Architect oligonucleotides and Donors oligonucleotides mixture of an endonuclease.

### Disclosed Aspects are Non-Limiting

While various aspects of the present invention have been shown and described herein, it is emphasized that such aspects are provided by way of example only. Numerous variations, changes and substitutions may be made without departing from the invention herein in its various aspects. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping unless clearly stated otherwise, it is intended that each such grouping provides the basis for and serves to identify various subset aspects, the subset aspects in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and aspects herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2^{nd} Ed. J. E. Bailey and D. F. Ollis, McGraw Hill, New York, 1986, e.g. Chapter 9, pages 533-657 for biological reactor design; Unit Operations of Chemical Engineering, 5th Ed., W. L. McCabe et al., McGraw Hill, New York 1993, e.g., for process and separation technologies analyses; Equilibrium Staged Separations, P. C. Wankat, Prentice Hall, Englewood Cliffs, NJ USA, 1988, e.g., for separation technologies teachings).

All publications, patents, and patent applications mentioned in this specification are entirely incorporated by reference.

### EXAMPLES

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Example 1: Architect Oligo Directed Fill in and Ligation

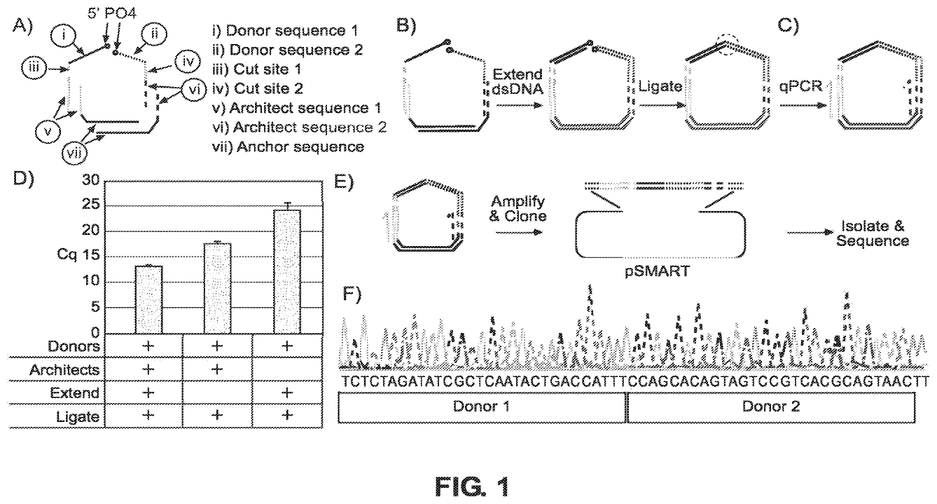

Referring now to FIG. 1, Architect oligos can be used to direct the ligation of two molecules of DNA. In this embodiment, a pair of Architect oligos consists of a complementary, overlapping 5′ sequence (the “Anchor” sequence (vii)) and unique 3′ sequences known as “Architect” sequences ((v) and (vi)). In this embodiment, Architect sequences are denoted by number (1 or 2) where each unique number refers to a unique Architect sequence and each Architect sequence consists of 25 nucleotides. It is appreciated however that the number of nucleotides of this sequence may be of any length, for example between 1 and 50000 nucleotides, or between 5 and 50 nucleotides. The “Anchor” sequence is such that pairs of Architect oligonucleotides can bind to each other if they contain complementary “Anchor” sequences. A pair of Architect oligonucleotides therefore has 3′ overhangs comprising the unique Architect sequences. These 3′ overhangs bind to and facilitate ligation of “Donor” oligonucleotides, wherein Donor oligonucleotides consist of a sequence complementary to a specific Architect sequence, an endonuclease targeting site ((iii) & (iv) hereafter referred to as Cut Site 1 or Cut Site 2), and include part of the sequence that is being synthesized (“Donor” DNA, (i) or (ii)). Donor oligonucleotides bind specific Architect sequences such that a 5′ overhang is created containing Cut Site 1 or 2 and the Donor DNA. In this embodiment, Cut Site 1 is 14 bp and Cut Site 2 is 13 bp and they are cleaved by Esp3i and SapI, respectively. The Donor DNA used in this embodiment is 30 bp. Therefore, the 5′ overhangs generated from Donor oligonucleotides binding to the Architect oligonucleotides are 43 to 44 bp. Furthermore, Donor oligonucleotides have 5′ phosphate groups. As shown in FIG. 1B, a pair of Architect oligonucleotides (referred to as Architect 1 and Architect 2) are connected by the Anchor sequence and each bind to Donor oligonucleotides (referred to as Donor 1 and Donor 2). A polymerase is then used to fill in the 5′ overhangs created by the Donor oligonucleotides binding the Architect oligonucleotides, such that the Donor DNA is now double-stranded DNA (dsDNA). This step is hereafter referred to as the “Extend” step. In FIG. 1B, a ligase is then used to join Donor 1 and Donor 2 such that the desired synthetic DNA (referred to as Donor 1-2) is created. As seen in FIGS. 1C and 1D, primers binding to Architect 1 and Architect 2 were used in a qPCR reaction to confirm these results. Compared to a negative control in which no Architect oligonucleotides were included and a negative control in which no polymerase was included in the Extend step, we see lower Cq values, indicating higher concentrations of the target (Donor 1-2). To further confirm this, we amplified and cloned Donor 1-2 and the surrounding Architects in order to isolate and sequence. As seen in FIG. 1F, sequencing yielded the desired sequence (as exemplified by SEQ ID NO: 15 in FIG. 1).

### Example 2: Multiplexed Architect Oligo Directed Fill in and Ligation

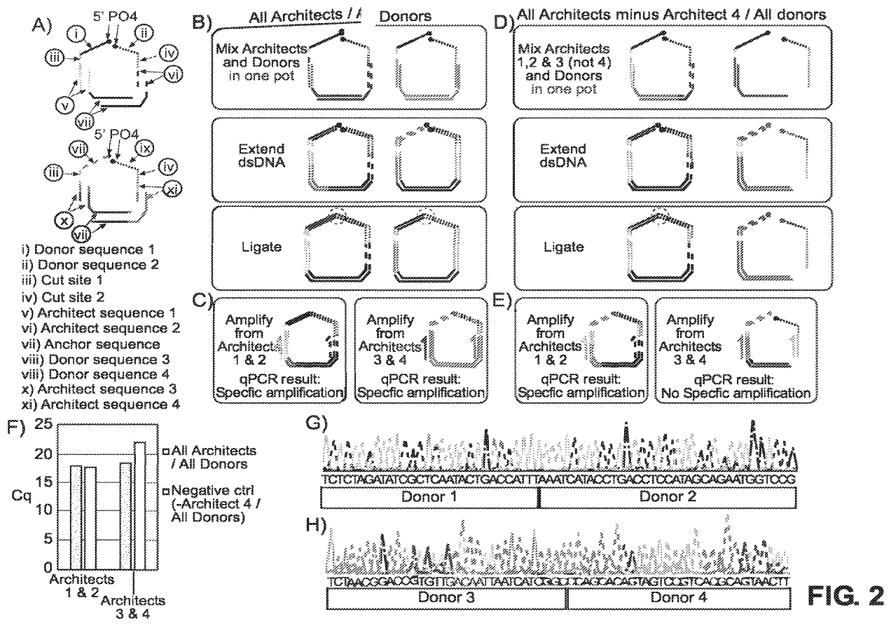

Architect oligonucleotides can be used to direct multiple different ligations in a single reaction. As seen in FIG. 2, two pairs of unique Architects to direct two different ligations in a single reaction. Architect 1 (v) and Architect 2 (vi) directed the ligation of Donor 1 (i) and Donor 2 (ii), while Architect 3 (x) and Architect 4 (xi) directed the ligation of Donor 3 (viii) and Donor 4 (ix) (FIG. 2B). These results by qPCR using primers that bind to the Architect sequences, as shown in FIG. 2C. A negative control in which we didn't add Architect 4, as illustrated in FIGS. 2D and 2E. qPCR results showed similar Cq values for Architect 1 and 2 in both reactions and similar Cq values for Architect 3 and 4 in the first reaction but higher Cq values in the control reaction, demonstrating how Architect oligonucleotides can be used in multiplex to improve ligation efficiencies (FIG. 2F). We further confirmed these results by amplifying and cloning the ligated sequences, as seen in FIGS. 2G (SEQ ID NO: 16) and 2H (SEQ ID NO: 17).

### Example 3: Multiple Rounds of Architect Oligo Directed Fill in and Ligation

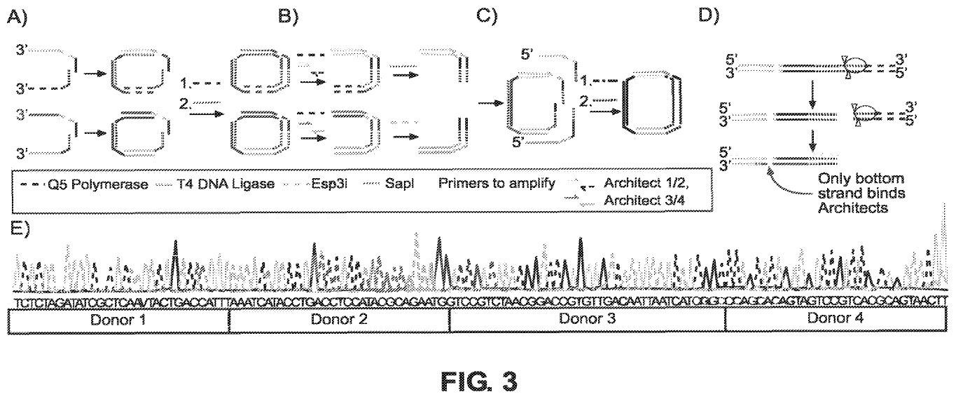

Following the steps in Example 2, we demonstrate that amplification of the ligated Donor DNA, followed by selective cleavage of either Cut Site 1 or 2 essentially creates a new Donor sequence that is double-stranded, includes the newly ligated sequences, and can be used in future rounds of Architect-directed ligations, thus demonstrating how multiple rounds of Architect-directed ligations can be used to synthesize DNA. In the first round, two pairs of Architect oligonucleotides were used to direct two different ligations, as seen in FIG. 3A. These ligated products were then amplified using primers that bind to the Architect sequences, creating dsDNA that contains both Architect sequences, Cut Site 1 and 2, and the newly ligated Donor sequences. For Architect pair 1 and 2, we used Esp3i to cleave Cut Site 2 such that Architect 2 and Cut Site 2 were removed, leaving Architect 1, Cut Site 1, and the newly ligated Donor DNA from the previous round (“Donor 1-2”). Similarly, for Architect pair 3 and 4, we used SapI to cleave Cut Site 1 such that Architect 3 and Cut Site 1 were removed, leaving Architect 4, Cut Site 2, and the newly ligated Donor DNA (“Donor 3-4”). The cleavage reactions were then combined with Architect 1 and Architect 4 for a second round of Architect-directed ligations. A polymerase in combination with two cycles of denaturing and extension was then used to Extend the Architect oligonucleotides once bound to the Donor sequences and the product of this was then ligated together to create “Donor 1-2-3-4”, as illustrated in FIG. 3C. To confirm this, we cloned the ligated product using Golden Gate Assembly, isolated colonies and sequenced. As seen in FIG. 3D, we confirmed that following two rounds of Architect-directed ligations we were able to go from four starting Donor sequences to a single joined product (as exemplified by SEQ ID NO: 18).

### Example 4: Architects with Covalently Linked 5′ Ends

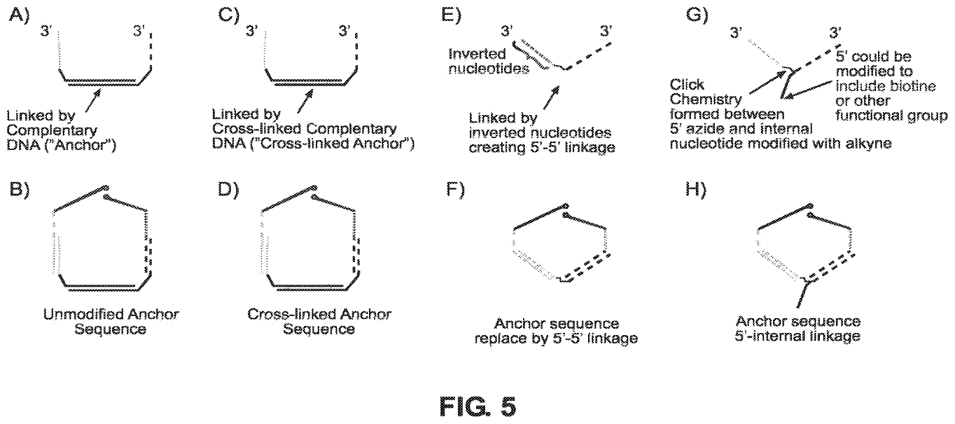

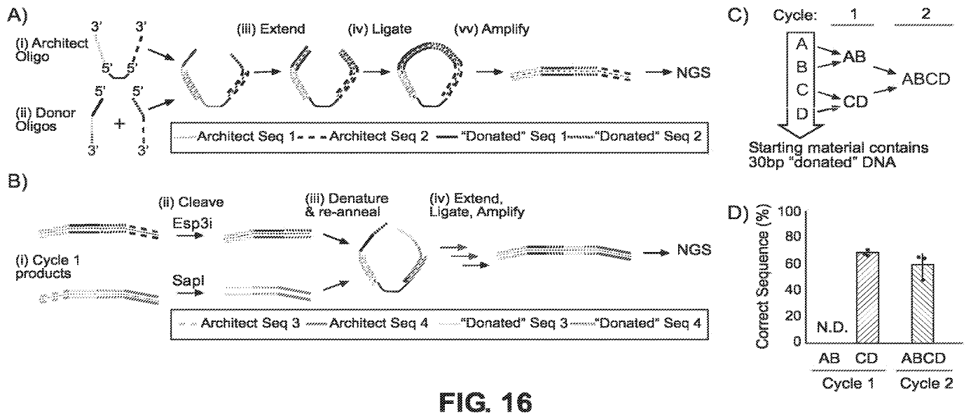

We demonstrate the use of single Architect oligonucleotide which encodes two Architect sequences but uses inverted nucleotides for one of the Architect sequences such that the two Architect sequences are linked by their 5′ terminus and the molecule has two 3′ termini (FIGS. 5E and 5F). Similar to Examples 1 and 3, we performed the Extend, Ligate, Amplify, and Cleave reactions of Cycle 1, starting with single-stranded donor oligos (FIG. 16A). We then completed a second cycle in which we combined two Cycle 1 products (FIG. 16B). In one of the first cycle products, we achieved 68.4+/−1.6% correct sequences and in the product of Cycle 2, we achieved 58.7+/−8.4% correct sequences (FIG. 16D).

### Example 5: Capture and Removal of Uncleaved and Unwanted Byproducts after the Cleavage Reaction Improves Cycle Efficiency

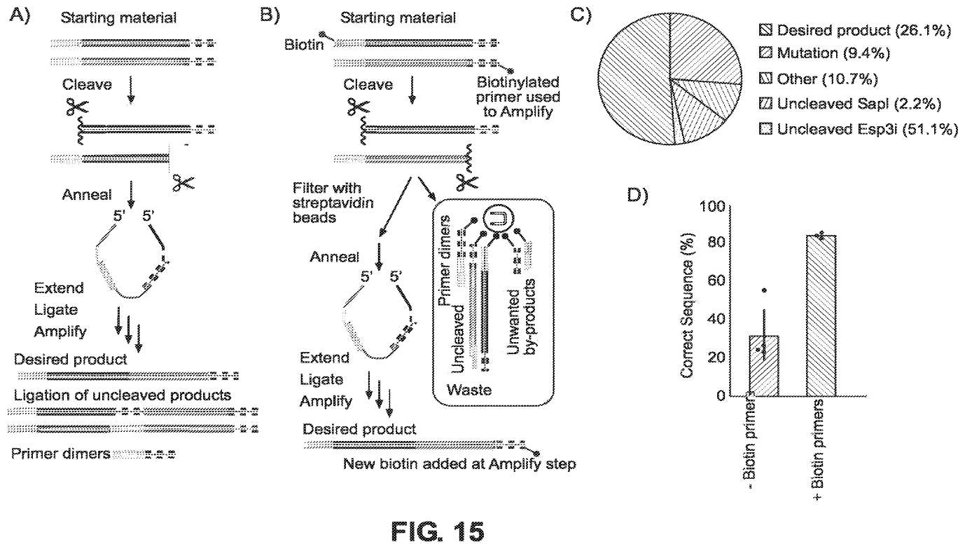

We demonstrate that use of a 5′ biotinylated primer with a non-biotinylated primer in the amplification step enables capture and removal of uncleaved products and other unwanted by-products after the cleavage step and before subsequent cycles. As illustrated in FIG. 15A, we started with two molecules of double-stranded DNA that resemble the products of an amplification step, encoding one unique Architect sequence on either end followed by unique cut sites, and with “donated” DNA in the middle. We then cleaved at one or the other cut site, thus creating subsequent cycle donors. These donors were combined with an Architect oligo, denatured and re-annealed in the presence of a polymerase, and then ligated. The ligation product was amplified and sequenced with an Illumina Next Generation Sequencing device. We compared this to starting with the same starting materials except containing a 5′ biotin on the side we planned to cleave (FIG. 15B). Following cleavage, streptavidin-coated magnetic beads were used to capture the biotinylated molecules, which included uncleaved products as well as unwanted by-products such as cleaved ends and primer dimers. As shown in FIG. 15C, the non-biotinylated starting material resulted in unwanted ligation products between cleaved and uncleaved donors as well as primer dimers that resulted in an average cycle efficiency of 32.3%+/−13.4% (FIG. 15D). With the addition of a biotinylated end, the uncleaved products were filtered out prior to the ligation, resulting in a cycle efficiency of 84.3%+/−1.4% (FIG. 15D).

### Example 6: Using Architect-Directed Ligations to Synthesize DNA from a Set of Standard Oligonucleotides

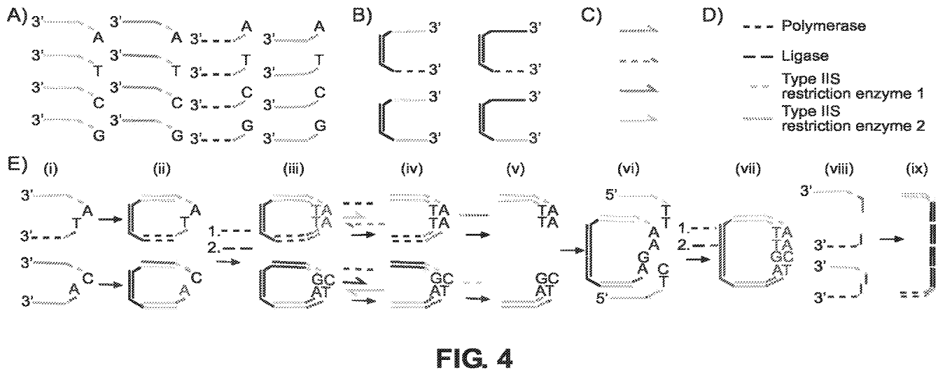

An embodiment wherein a standardized set of Architect and Donor oligonucleotides, in combination with standardized primers and enzymes, can enable synthesis of any sequence of DNA. In the example outlined in FIG. 4, 16 Donor oligonucleotides and 4 Architect oligonucleotides are used as starting material. The 16 Donor oligonucleotides consist of one of four Architect sequences at the 3′ end, one of two Cut Sites, and one of four single nucleotides at the 5′ end such that all combinations of Architect sequences are matched with all combinations of Donor sequences (FIG. 4A). Additionally, these Donor oligonucleotides have a 5′ phosphate. The 4 Architect oligonucleotides can be grouped into 4 sets of Architect pairs bound together by the Anchor sequence: Architect 1 and Architect 3 can both bind to Architect 2 or Architect 4 (FIG. 4B). These oligonucleotides can be used to synthesize DNA in combination with a set of standard primers that bind to the Architect sequences (FIG. 4C) and a set of enzymes, including a polymerase, ligase, and enzymes for cutting the DNA (FIG. 4D). As illustrated in FIG. 4E, the synthesis would initiate with the selection of the appropriate Donor oligonucleotides in combination with Architect pairs to synthesize the example sequence 5′-TTGA-3′. As described in Example 1, 2, and 3, the overhangs generated by Donor oligonucleotides binding to the Architects would be filled in by a polymerase and then the double-stranded DNA would be ligated together to join the Donor sequences, creating 5′-TT-3′ and 5′-GA-3′. A PCR reaction using the primers for each Architect pair would then specifically amplify the ligated products. The product containing the sequence 5′-TT-3′ would be cleaved at Cut Site 2 while the product containing the sequence 5′-GA-3′ would be cleaved at Cut Site 1. As described in Example 3, this would leave sequences containing an Architect sequence, a Cut Site, and the newly ligated Donor sequences. In the presence of a new Architect pair and a polymerase, the double-stranded cleavage products would be mixed, denatured and, when the Donor DNA anneals to the Architects, the overhangs would be extended to generate double strand DNA. This would then be ligated together, creating the newly synthesized sequence 5′-TTGA-3′. For longer sequences, the product would then be amplified, cleaved at either Cut Site 1 or 2, and used in combination with other newly synthesized Donor DNA to generate the desired synthesized DNA. In this way, two rounds of the steps described here would generate a 16 bp sequence and three rounds would generate a 64 bp sequence, illustrating the use of this method to exponentially synthesize DNA. This method is not meant to be limiting and could be done with 2 or more Architect sequences, using Donor oligonucleotides contributing more than one nucleotide of Donor sequence, using Architect pairs that are operably linked at their 5′ ends (FIG. 5), using other methods to cleave DNA (Example 8), using alternatives to the amplification step described here (Example 9 and 10), and using different methods to move DNA from one reaction to another and other approaches obvious to one skilled in the arts.

### Example 7: Architects are Covalently Linked at the 5′ Ends

An embodiment wherein the Anchor sequence of each Architect pair is cross-linked using interstrand cross links such that the strands cannot be denatured (FIGS. 5C and 5D).

An embodiment wherein a pair of Architect sequences is linked covalently (such as using click chemistry) between the 5′ terminus of one Architect (Architect 1) and an internal modified nucleotide of the other Architect (Architect 2), such that the 5′ terminus of Architect 2 could be modified with a purification tag or other modification (FIGS. 5G and 5H).

### Example 8: Different Methods of Cleaving DNA

An embodiment wherein Cut Site 1 and 2 are cleaved by Type IIS restriction enzymes, as shown in Example 3 where Cut Site 1 is cleaved by SapI and Cut Site 2 is cleaved by Esp3i.

An embodiment wherein Cut Site 1 and 2 are cleaved by Type IIS nicking restriction enzymes such that the future Donor strand of the DNA is cleaved but not the non-Donor strand. This method could be used in combination with denaturing and capture as described in Example 10.

An embodiment wherein Cut Site 1 and 2 are cleaved by a CRISPR endonuclease or mutant thereof wherein 1) cleavage is outside of the crRNA target sequence and 2) the enzyme has limited or no non-specific endonuclease activity.

An embodiment wherein Cut Site 1 and 2 are removed and the Architect sequences contain Cas12a PAM sites such that unique Cas12a gRNA can be used to cleave the target. In this embodiment, the PAM sites in the Architect are located 14 bp from the end of the Architect that joins the Donor DNA and a 14 bp crRNA is used to sequence specifically cleave the DNA such that the non-target strand is cleaved 14 bp from the PAM. (Lei et al. 2017; Lynch, Moreb, and Yang 2021) In this embodiment, the target strand cleavage is 22 bp away from the PAM and therefore in the Donor DNA. However, the 5′ overhang generated in the Donor DNA by this cleavage reaction is filled in by the polymerase in the subsequent Extend step.

An embodiment wherein the Cas12a nuclease is mutated such that it cleaves one strand selectively.

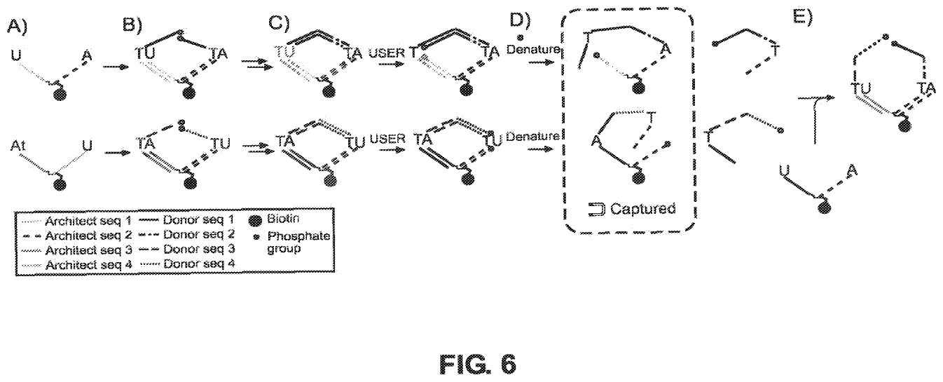

An embodiment wherein Cut Site 1 and 2 are removed and an uracil may selectively be inserted into the 5′ end of the Architect sequence, as shown in FIG. 6. This method would require a polymerase that can incorporate uracil, such as but not limited to Q5 Polymerase by NEB (NEB #M0515L). By selectively including a uracil at one end of the Architect sequence but not the other, USER enzyme or a combination of UDG and Endonuclease VIII could be used to excise the uracil, effectively cleaving a single strand of the DNA in the same manner as a nicking endonuclease.

### Example 9: Asymmetric PCR to Generate ssDNA for Moving Donor DNA to New Reactions

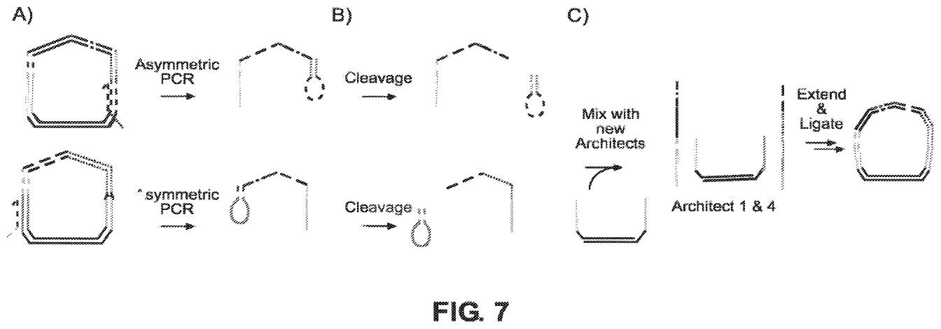

An embodiment wherein the amplification step in Example 3 and 4 uses asymmetric PCR to generate single stranded DNA (ssDNA) and the primer at the 5′ end of the ssDNA contains a 5′ tail encoding a sequence complementary to the Architect or Cut Site such that a hairpin forms at the 5′ end (FIG. 7A). Further, this hairpin enables cleaving the DNA such that only single stranded Donor DNA is left at the 5′ end (FIG. 7B), and the remaining single-stranded DNA can act as Donor oligonucleotide in subsequent rounds of Architect directed ligations (FIG. 7C). DNA cleavage could be done with restriction enzymes, CRISPR endonucleases, or USER enzyme as described in Example 8. This method is not meant to be limiting and could be modified in various ways obvious to one skilled in the arts, including using two primers in asymmetric ratios instead of one primer to preferentially generate single stranded DNA.

### Example 10: Moving DNA Between Cycles without Amplification

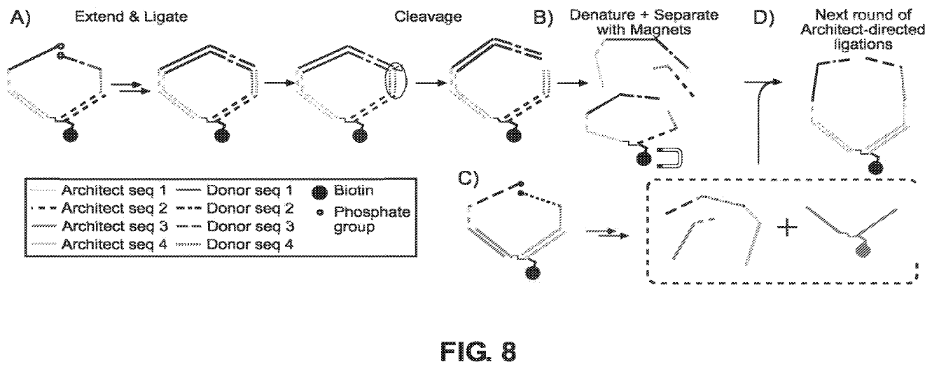

An embodiment wherein the generation of Donor DNA using amplification (as described in Example 3) is replaced with denaturing and isolating single-stranded DNA, as described in FIG. 8. In this example, the Architect pair includes a purification tag (eg., biotin) and directs the ligation of Donor sequences, as described in Example 3 and 4 (FIG. 8A). Following ligation, either Cut Site 1 or Cut Site 2 is cleaved and the DNA is denatured using heat or alkaline conditions. Under these conditions, the Architect pair are captured and removed using the purification tag, such that the remaining single-stranded DNA includes at least one new Donor DNA molecule encoding an Architect sequence, Cut Site, and the newly ligated Donor DNA (FIG. 8B). In a subsequent reaction, two such Donor molecules are introduced with a new pair of Architect sequences to begin the next Architect-directed ligation reaction (FIG. 8D). This method is not meant to be limiting and you could use different Architect types as described in Example 7, as well as other combining this with other approaches known to those skilled in the arts.

### Example 11: Using Architects to Direct Single-Stranded Ligations

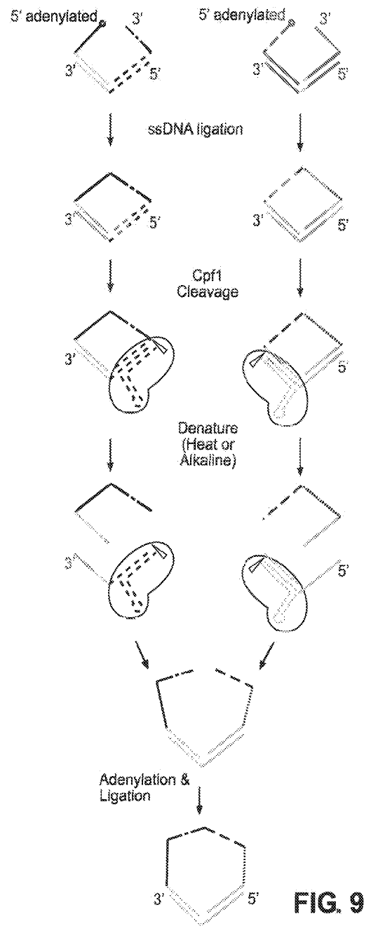

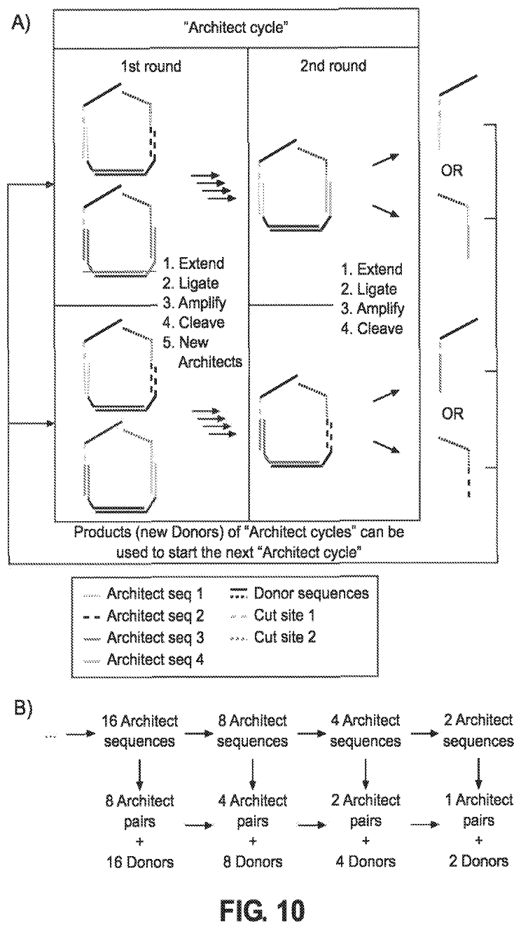

An embodiment wherein a pair of Architect sequences are encoded in a single oligo containing one Architect at the 5′ end and the other Architect at the 3′ end. In this example, the Donor oligonucleotides would contain Architect binding sequences at their 5′ and 3′ ends, respectively. The Donor sequences would then be ligated together using a single-stranded DNA ligase (such as AppLigase) that joins the 5′ pre-adenylated end of one Donor with the 3′ OH group of the other Donor (FIG. 9). As shown in FIG. 9, Architect 1 and Architect 2 may be operably linked in a single oligonucleotide. While in some aspects, there are no additional nucleotides contiguous and therebetween Architect 1 and Architect 2. However, it is appreciated that aspects of the invention encompass a single oligonucleotide containing two distinct Architect sequences with any number of nucleotides between the two architect sequences and contiguous with both architect sequences. This method is not meant to be limiting and could be modified to incorporate other methods known by one skilled in the arts, including the use of other ligases or methods of denaturing and purifying single-stranded DNA.

### Example 12: Block Polymerase to Reduce Amplification of the Anchor Sequence

An embodiment wherein Architect oligonucleotides containing an Anchor sequence (such as Examples 1-4) also contains one or more modified nucleotides between the Architect sequence and the Anchor sequence such that a polymerase copying this strand would be blocked from copying the Anchor sequence. The modified nucleotide could include but is not limited to one or more uracil, inverted nucleotides, or modifications that would sterically block the progression of the polymerase such as a biotin or carbon spacer.

TABLE 1
Example 13: Comparing how Architect-directed ligations
reduce the number of reactions required.

| | | Parallel | Parallel | Longest |
|---|---|---|---|---|
| | Parallel | reactions | reactions | synthesized |
| | Ligations | (4 Architects) | (16 Architects) | DNA (bp) |
| Round 1 | 512 | 256 | 64 | 2 |
| Round 2 | 256 | 128 | 32 | 4 |
| Round 3 | 128 | 64 | 16 | 8 |
| Round 4 | 64 | 32 | 8 | 16 |
| Round 5 | 32 | 16 | 4 | 32 |
| Round 6 | 16 | 8 | 2 | 64 |
| Round 7 | 8 | 4 | 1 | 128 |
| Round 8 | 4 | 2 | 1 | 256 |
| Round 9 | 2 | 1 | 1 | 512 |
| Round 10 | 1 | 1 | 1 | 1024 |
| Number | 1023 | 512 | 130 | |
| of wells | | | | |
| total: | | | | |

### Example 14: Amplification of Final Synthetic DNA Sequence

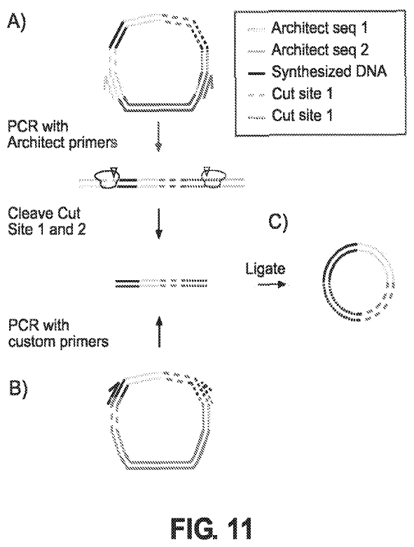

The final synthesized DNA sequence is amplified after the final Architect-directed ligation by using primers that bind to the Architect sequences and amplify the synthesized DNA (FIG. 11A). Subsequent cleavage of Cut Site 1 and Cut Site 2 releases the synthesized DNA with 5′ overhangs that can be filled in using a polymerase. The synthetic DNA could then be purified using multiple methods known to one skilled in the arts, such as column-based purifications, bead-based approaches, or HPLC.

An embodiment wherein the final synthesized DNA sequence is amplified after the final Architect-directed ligation by using primers specific to the synthesized sequence (FIG. 11B).

An embodiment wherein the final synthesized DNA sequence includes a plasmid origin of replication and antibiotic resistance gene and is self-ligated following amplification such that it can be transformed into E. coli or other organism for further propagation or use. (FIG. 11C).

### Example 15: Exponential Generation of a DNA Sequence

The Architect-directed ligations are used to linearly generate the desired sequence of DNA by repeatedly adding Donor oligonucleotides to a growing strand of DNA.

An embodiment wherein Architect-directed ligations are used to exponentially generate the desired sequence of DNA by repeatedly combining products of Architect-directed ligations such that the length of the product DNA doubles after each round of Architect-directed ligations.

### Example 16: Generation of Donor DNA

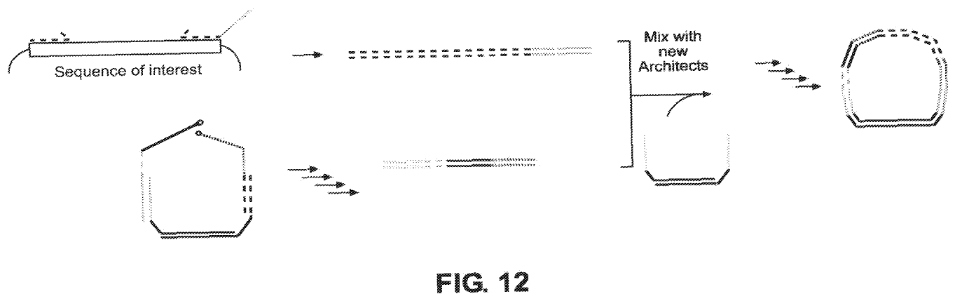

A PCR step can be used to generate Donor DNA out of any existing template by incorporating a Cut Site and Architect sequence into the 5′ end of one of the primers used to amplify the target DNA (FIG. 12). By selecting which primer to incorporate the Architect sequence with, the orientation of the subsequent Architect-directed ligation can be controlled.

### Example 17: A Mixture of Donor Oligonucleotides

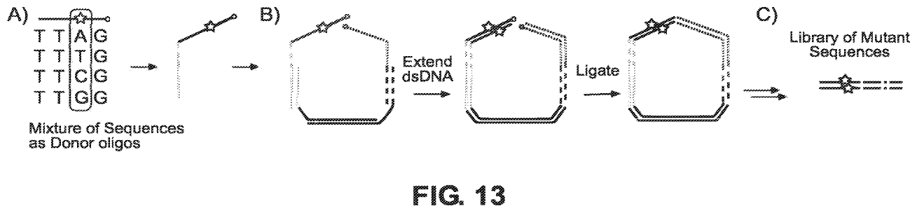

A mixture of Donor oligonucleotides could be used to generate a targeted mutant library as the synthesized DNA (FIG. 13).

### Example 18: Polymerases Useful in a DNA Amplification Method

The polymerase used does not leave a terminal adenine, such as Q5 Polymerase, Q5U Polymerase, Phusion Polymerase, and Vent Polymerase.

### Example 19: Ligases Useful in a DNA Amplification Method

The ligase used can join blunt ends of DNA, such as T4 DNA ligase, T3 DNA ligase or any mutants thereof.

### Example 20: Use of Purification Tags

One or more enzymes used can be covalently linked to a biotin or other purification tag such that enzymes can be removed and/or reused upon completion of the reaction. One or more Architect oligonucleotides can contain an internal or terminal biotin or other purification tag such that the Architects can be captured and separated from other molecules of single or double stranded DNA or from the enzymes. One or more of the Donor oligonucleotides can contain an internal or terminal biotin or other purification tag such that the Donor oligonucleotides can be captured and separated from other molecules of single or double stranded DNA or from the enzymes. One or more of the primers used for amplification can contain an internal or terminal biotin or other purification tag such that the Donor oligonucleotides can be captured and separated from other molecules of single or double stranded DNA or from the enzymes.

### Example 21: Length of Donor Oligonucleotides not Limited

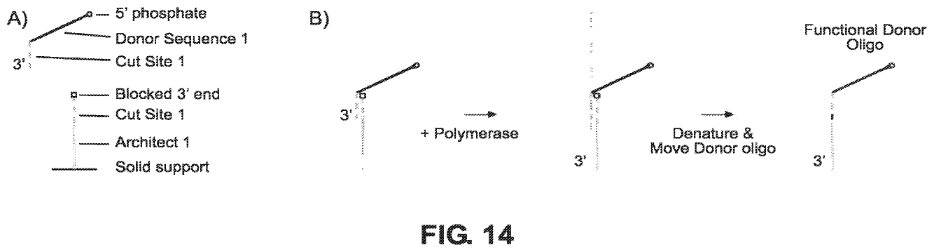

The starting material for synthesis can include shorter Donor oligonucleotides containing the Cut Site sequence (1 or 2) at the 3′ end of the Donor oligonucleotide and the Donor sequence, including a 5′ phosphate group, at the 5′ end. In order to create the full-length Donor oligonucleotides described in Example 1-3, a method to extend the 3′ end of the oligonucleotide and thus incorporate the Architect sequence could include: another oligonucleotide (the “Template”) attached to a solid support or biotin at the 5′ end, with a blocking group at the 3′ end (eg., dideoxyribonucleotides), and encoding an Architect sequence at the 5′ end and the same Cut Site sequence (complementary to the Cut Site sequence on the Donor oligonucleotide) at the 3′ end (FIG. 14). The Donor oligonucleotide could bind to the Template oligonucleotide via the overlapping Cut Site sequence in the presence of a polymerase such that the polymerase extends the 3′ end of the Donor oligonucleotide, copying the sequence of the Architect. The sequence could then be denatured, releasing and purifying the Donor oligonucleotide from the immobilized/captured Template oligonucleotide. This method could be incorporated prior to the first round of Architect-directed ligations to reduce the number of starting materials required.

### Example 22: Recycling of Materials

The Architect oligonucleotides can be reused. In this example, the Architect oligonucleotides include a biotin tag or other purification tag allowing capture of these oligonucleotides following Architect-directed ligations. After capture, Cut Site 1 and Cut Site 2 are cleaved, removing any Donor DNA from the immobilized Architects. Additionally, the remaining DNA is denatured and washed such that only the original strand of Architect oligonucleotides remains immobilized. The Architects are then ready to be reused for future Architect-directed ligations.

### Example 23: Exonuclease Useful in a DNA Amplification Method

An exonuclease, such as Exonuclease I (from E. coli), can be used to selectively degrade single-stranded DNA. This could be used to degrade excess Donor oligonucleotide following Architect-directed ligations.

### Materials & Methods

The initial “Extend” step used Q5 High Fidelity 2× Master Mix (New England Biolabs (NEB), M0429S) in a 10 uL reaction. The desired Architect oligonucleotides (selected from EM01-EM04 in Table 1) were pre-mixed at 0.1 uM and then added to the reaction at a final concentration of 0.01 uM. Corresponding Donor oligonucleotides (selected from EM05-EM08 in Table 1) were added at a final concentration of 0.02 uM. The reaction was then run in a PCR machine using the following thermocycling conditions: 72 C for 1 minute, 98 C for 10 seconds, 72 C for 10 seconds, 98 C for 10 seconds, 72 C for 40 seconds.

Following the “Extend” step, a ligation reaction was set up using 1 uL from the Extend step reaction, 1 uL of 10×T4 DNA Ligase Buffer, 1 uL of T4 DNA Ligase (NEB, M0202S) and 7 uL of water. This reaction was then left at room temperature for 15 minutes, followed by 10 minutes at 65 C to denature the ligase.

After denaturing, the ligase mixture was diluted by adding 90 uL of water. 1 uL of the diluted reaction was then used in a PCR reaction with Q5 High Fidelity 2× Master Mix in a 20 uL reaction using primers that bind to the Architect sequences (selected from EM09-EM12 in Table 1). The PCR thermocycling protocol was as follows: 98 C for 2 minutes, 30 cycles of 98 C for 10 seconds, 64 C for 10 seconds, and 72 C for 10 seconds, followed by 72 C for 2 minutes and a hold at 10 C once completed.

For cleavage reactions, 2 uL of PCR product was added to a 20 uL reaction containing 1 uL of either Esp3i (NEB, R0734S) or SapI (NEB, R0569S), 2 uL of CutSmart buffer, and 15 uL of water. The reaction was placed at 37 C for 45 minutes, followed by denaturing at 65 C for 15 minutes.

For subsequent “Extend” steps, 2 uL of Donor DNA cleaved by Esp3i was mixed with 2 uL of Donor DNA cleaved by SapI, 1 uL of Architect oligonucleotides (final concentration 0.01 uM), and 5 uL of Q5 High Fidelity 2× Master Mix (NEB, M0429S). The reaction was then run in a PCR machine (name x) using the following thermocycling conditions: 72 C for 1 minute, 98 C for 10 seconds, 72 C for 10 seconds, 98 C for 10 seconds, 72 C for 40 seconds. Subsequent ligation and cleavage reactions were as described above.

qPCR was used to measure ligation activity using primers that bind to the Architect sequences (EM09-EM12 in Table 1) and amplify across the Donor sequences. Luna Universal qPCR Master Mix (NEB, M3003S) was used with a Chai Bio Open qPCR machine. The thermocycling protocol was as follows: 95 C for 30 seconds followed by 40 cycles of 95 C for 30 seconds, 60 C for 30 seconds.

For cloning and sequencing Donor sequences, primers EM09-EM12 were used (depending on the flanking Architect sequences) to amplify the ligated target using Q5 High Fidelity 2× Master Mix in a 50 uL reaction. The product was then purified and resuspended in 30 uL of water using Zymo DNA Clean & Concentrator-25 (Zymo Research, D4033). Similarly, the pSMART backbone (Lucigen, #40704-2) was amplified and purified with primers EM13 and EM14. 2 uL of the purified pSMART DNA was mixed with 3 uL of purified Donor DNA, 2 uL of 10×T4 DNA ligase buffer, 0.5 uL of T4 DNA ligase (NEB, M0202S), 0.5 uL of BsaI-HF V2 (NEB, R3733S), and 12 uL of water. The reaction was then run in a PCR machine using the following thermocycling protocol: 10 cycles of 37 C for 5 minutes followed by 16 C for 5 minutes, and then a final step at 37 C for 5 minutes followed by a denaturing step at 80 C for 5 minutes and a hold at 10 C. This was then transformed into Ecloni 10G electrocompetent cells (Lucigen, #60117-1) and plated on agar plates containing kanamycin. For sequencing, colony PCR with Econotaq PLUS 2× Master Mix (Lucigen, #30035-1) was used to amplify from the pSMART backbone using primers EM13 and EM14 and samples were sent for Sanger sequencing at GENEWIZ.

To test 5′ linked Architects, the same protocols described above were used except that the pair of Architects described above were instead ordered from IDT (Coralville, Iowa) with one Architect sequence ordered as inverted nucleotides such that both Architect sequences were covalently linked at their 5′ ends and the molecule has two 3′ ends (EM20-EM22). The donor oligos used were EM23-EM26.

For measuring single cycle efficiency starting with double-stranded DNA, DNA was amplified from amilCP chromoprotein (Addgene: 117847) using primers EM15 and EM16 or EM17 and EM18. A 5′ biotin was added to EM16 and EM17 to make biotinylated donor DNA. The “Cleavage” reaction was as described above but was incubated at 37 C for 4 hours. The donor amplified by EM15/EM16 was cleaved with Esp3i while the donor amplified by EM17/EM18 was cleaved by SapI. For the biotinylated DNA, streptavidin (catalogue number) binding was adapted from the manufacturer's protocol. Briefly, 1 uL of streptavidin was washed 3 times in B&W buffer, resuspended in 2 uL of B&W and added to the “Cleavage” reaction prior to denaturing the Type IIS enzymes. Samples were mixed well, denatured at 65 C for 15 minutes, and then placed on a magnetic rack. The supernatant was carried forward into “Extend” using a 5′ linked Architect oligonucleotide (EM22) and “Ligate” as previously described. Experiments were performed in triplicate and amplified with primers EM19-EM23 were amplified and samples were sent for NGS sequencing using Azenta's AMP-EZ service.

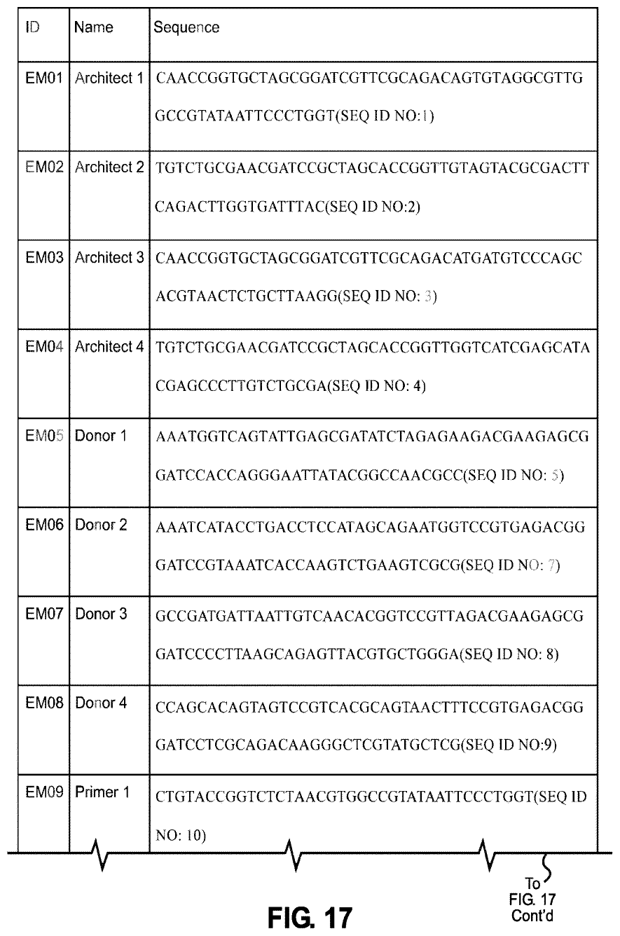

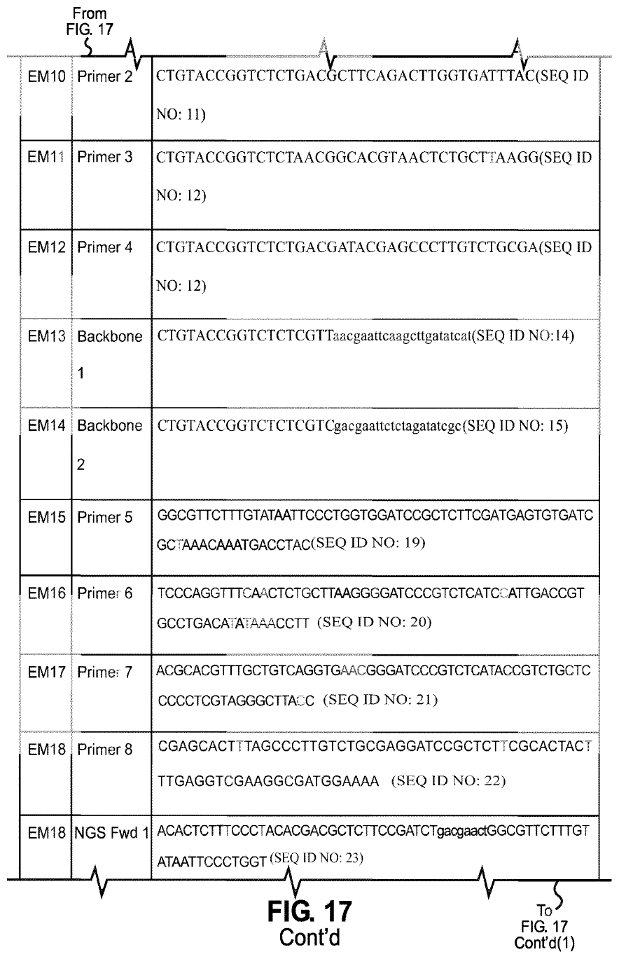

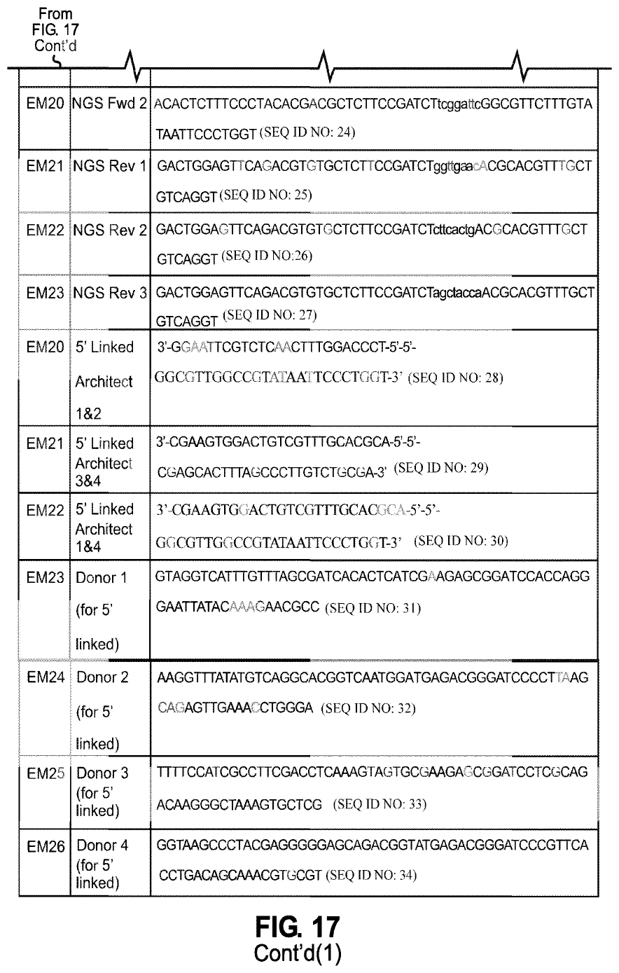

FIG. 17 provides a table of oligonucleotide sequences used in these methods.

Unless otherwise stated, all materials and reagents were of the highest grade possible.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A mixture comprising:
a. a pair of Architect oligonucleotides that are partially complimentary to each other, each Architect oligonucleotide comprising:
an anchor sequence operably linked to
an architect sequence; and
b. at least one Donors oligonucleotide comprising:
an architect complimentary sequence operably linked to a
a cleavage site that is operatively linked to a
donor sequence 5′ overhang of at least 1 nt.

2. The mixture of claim 1 wherein one of the Architect or Donor oligonucleotides further comprise a purification tag.

3. The mixture of claim 1 wherein a pair of Architect oligonucleotides are operably linked to each other covalently.

4. The mixture of claim 1 wherein any Architect oligonucleotide comprises a modification rendering the oligonucleotide resistant to endonuclease cleavage.

5. The mixture of claim 1 comprising two donor oligonucleotides.

6. The mixture of claim 1 further comprising a DNA polymerase.

7. The mixture of claim 6, wherein the DNA polymerase lacks a 5′-3′ nuclease activity.

8. The mixture of claim 6, wherein the DNA polymerase lacks strand displacement activity.

9. The mixture of claim 6, wherein the DNA polymerase is thermostable.

10. The mixture of claim 1 further comprising a ligase.

11. The mixture of claim 10, wherein the ligase is thermostable

12. The mixture of claim 1 further comprising an endonuclease.

13. The mixture of claim 12 wherein the endonuclease is thermostable.

14. The method of claim 12, wherein the endonuclease is a CRISPR or type IIS endonuclease.

15. The mixture of claim 1 further comprising UDG enzyme.

16. A template independent, exponential method of synthesizing an oligonucleotide product comprising:
providing a pair of Architect oligonucleotides that are partially complimentary to each other, each Architect oligonucleotide comprising:
an anchor sequence operably linked to
an architect sequence;
providing a panel of Donors oligonucleotides, each donor oligonucleotide comprising:
an architect complimentary sequence operably linked to a
a cleavage site that is operatively linked to a
a subsequence of the oligonucleotide product;
mixing the Architect oligonucleotides and Donors oligonucleotides;
conducting on the Architect oligonucleotides and Donors oligonucleotides mixture repeated cycles of Extend, Ligation, Amplification, and Cleavage, wherein each cycle enlarges the subsequence of the oligonucleotide product until the subsequence represents the complete oligonucleotide product.

17. The method of claim 16, wherein the two pair of Architect oligonucleotides are provided.

18. The method of claim 16, wherein Extend comprises addition to the Architect oligonucleotides and Donors oligonucleotides mixture of a polymerase.

19. The method of claim 16, wherein Ligation comprises addition to the Architect oligonucleotides and Donors oligonucleotides mixture of a ligase.

20. The method of claim 16, wherein Amplification comprises addition to the Architect oligonucleotides and Donors oligonucleotides mixture of architect complimentary primers and application of PCT suitable conditions to the mixture.

21. The method of claim 16, wherein Cleavage comprises addition to the Architect oligonucleotides and Donors oligonucleotides mixture of an endonuclease.


SEQUENCE LISTING (34 sequences)

SEQ ID NO: 1 - DNA, 60 bp, synthetic construct
  misc_feature 1..60: note = Synthetic
  source 1..60: mol_type = other DNA
caaccggtgctagcggatcgttcgcagacagtgtaggcgttggccgtataattccctggt

SEQ ID NO: 2 - DNA, 60 bp, synthetic construct
  misc_feature 1..60: note = Synthetic
  source 1..60: mol_type = other DNA
tgtctgcgaacgatccgctagcaccggttgtagtacgcgacttcagacttggtgatttac

SEQ ID NO: 3 - DNA, 60 bp, synthetic construct
  misc_feature 1..60: note = Synthetic
  source 1..60: mol_type = other DNA
caaccggtgctagcggatcgttcgcagacatgatgtcccagcacgtaactctgcttaagg

SEQ ID NO: 4 - DNA, 60 bp, synthetic construct
  misc_feature 1..60: note = Synthetic
  source 1..60: mol_type = other DNA
tgtctgcgaacgatccgctagcaccggttggtcatcgagcatacgagcccttgtctgcga

SEQ ID NO: 5 - DNA, 72 bp, synthetic construct
  misc_feature 1..72: note = Synthetic
  source 1..72: mol_type = other DNA
aaatggtcagtattgagcgatatctagagaagacgaagagcggatccaccagggaattatacggccaacgcc

SEQ ID NO: 6 - DNA, 72 bp, synthetic construct
  misc_feature 1..72: note = Synthetic
  source 1..72: mol_type = other DNA
aaatcatacctgacctccatagcagaatggtccgtgagacgggatccgtaaatcaccaagtctgaagtcgcg

SEQ ID NO: 7 - DNA, 72 bp, synthetic construct
  misc_feature 1..72: note = Synthetic
  source 1..72: mol_type = other DNA
gccgatgattaattgtcaacacggtccgttagacgaagagcggatccccttaagcagagttacgtgctggga

SEQ ID NO: 8 - DNA, 72 bp, synthetic construct
  misc_feature 1..72: note = Synthetic
  source 1..72: mol_type = other DNA
ccagcacagtagtccgtcacgcagtaactttccgtgagacgggatcctcgcagacaagggctcgtatgctcg

SEQ ID NO: 9 - DNA, 38 bp, synthetic construct
  misc_feature 1..38: note = Synthetic
  source 1..38: mol_type = other DNA
ctgtaccggtctctaacgtggccgtataattccctggt

SEQ ID NO: 10 - DNA, 38 bp, synthetic construct
  misc_feature 1..38: note = Synthetic
  source 1..38: mol_type = other DNA
ctgtaccggtctctgacgcttcagacttggtgatttac

SEQ ID NO: 11 - DNA, 38 bp, synthetic construct
  misc_feature 1..38: note = Synthetic
  source 1..38: mol_type = other DNA
ctgtaccggtctctaacggcacgtaactctgcttaagg

SEQ ID NO: 12 - DNA, 38 bp, synthetic construct
  misc_feature 1..38: note = Synthetic
  source 1..38: mol_type = other DNA
ctgtaccggtctctgacgatacgagcccttgtctgcga

SEQ ID NO: 13 - DNA, 41 bp, synthetic construct
  misc_feature 1..41: note = Synthetic
  source 1..41: mol_type = other DNA
ctgtaccggtctctcgttaacgaattcaagcttgatatcat

SEQ ID NO: 14 - DNA, 39 bp, synthetic construct
  misc_feature 1..39: note = Synthetic
  source 1..39: mol_type = other DNA
ctgtaccggtctctcgtcgacgaattctctagatatcgc

SEQ ID NO: 15 - DNA, 60 bp, synthetic construct
  misc_feature 1..60: note = Synthetic
  source 1..60: mol_type = other DNA
tctctagatatcgctcaatactgaccatttccagcacagtagtccgtcacgcagtaactt

SEQ ID NO: 16 - DNA, 64 bp, synthetic construct
  misc_feature 1..64: note = Synthetic
  source 1..64: mol_type = other DNA
tctctagatatcgctcaatactgaccatttaaatcatacctgacctccatagcagaatggtccg

SEQ ID NO: 17 - DNA, 63 bp, synthetic construct
  misc_feature 1..63: note = Synthetic
  source 1..63: mol_type = other DNA
tctaacggaccgtgttgacaattaatcatcggcccagcacagtagtccgtcacgcagtaactt

SEQ ID NO: 18 - DNA, 127 bp, synthetic construct
  misc_feature 1..127: note = Synthetic
  source 1..127: mol_type = other DNA
tctctagatatcgctcaatactgaccatttaaatcatacctgacctccatagcagaatggtccgtctaacggaccgtgttgacaattaatcatcggcccagcacagtagtccgtcacgcagtaactt

SEQ ID NO: 19 - DNA, 69 bp, synthetic construct
  source 1..69: mol_type = other DNA
ggcgttctttgtataattccctggtggatccgctcttcgatgagtgtgatcgctaaacaaatgacctac

SEQ ID NO: 20 - DNA, 68 bp, synthetic construct
  source 1..68: mol_type = other DNA
tcccaggtttcaactctgcttaaggggatcccgtctcatccattgaccgtgcctgacatataaacctt

SEQ ID NO: 21 - DNA, 68 bp, synthetic construct
  source 1..68: mol_type = other DNA
acgcacgtttgctgtcaggtgaacgggatcccgtctcataccgtctgctccccctcgtagggcttacc

SEQ ID NO: 22 - DNA, 69 bp, unidentified
  source 1..69: mol_type = other DNA
cgagcactttagcccttgtctgcgaggatccgctcttcgcactactttgaggtcgaaggcgatggaaaa

SEQ ID NO: 23 - DNA, 66 bp, synthetic construct
  source 1..66: mol_type = other DNA
acactctttccctacacgacgctcttccgatctgacgaactggcgttctttgtataattccctggt

SEQ ID NO: 24 - DNA, 66 bp, synthetic construct
  source 1..66: mol_type = other DNA
acactctttccctacacgacgctcttccgatcttcggattcggcgttctttgtataattccctggt

SEQ ID NO: 25 - DNA, 60 bp, synthetic construct
  source 1..60: mol_type = other DNA
gactggagttcagacgtgtgctcttccgatctggttgaacacgcacgtttgctgtcaggt

SEQ ID NO: 26 - DNA, 60 bp, synthetic construct
  source 1..60: mol_type = other DNA
gactggagttcagacgtgtgctcttccgatctcttcactgacgcacgtttgctgtcaggt

SEQ ID NO: 27 - DNA, 60 bp, synthetic construct
  source 1..60: mol_type = other DNA
gactggagttcagacgtgtgctcttccgatctagctaccaacgcacgtttgctgtcaggt

SEQ ID NO: 28 - DNA, 50 bp, synthetic construct
  source 1..50: mol_type = other DNA
ggaattcgtctcaactttggaccctggcgttggccgtataattccctggt

SEQ ID NO: 29 - DNA, 50 bp, synthetic construct
  source 1..50: mol_type = other DNA
cgaagtggactgtcgtttgcacgcacgagcactttagcccttgtctgcga

SEQ ID NO: 30 - DNA, 50 bp, synthetic construct
  source 1..50: mol_type = other DNA
cgaagtggactgtcgtttgcacgcaggcgttggccgtataattccctggt

SEQ ID NO: 31 - DNA, 69 bp, synthetic construct
  source 1..69: mol_type = other DNA
gtaggtcatttgtttagcgatcacactcatcgaagagcggatccaccagggaattatacaaagaacgcc

SEQ ID NO: 32 - DNA, 68 bp, synthetic construct
  source 1..68: mol_type = other DNA
aaggtttatatgtcaggcacggtcaatggatgagacgggatccccttaagcagagttgaaacctggga

SEQ ID NO: 33 - DNA, 69 bp, synthetic construct
  source 1..69: mol_type = other DNA
ttttccatcgccttcgacctcaaagtagtgcgaagagcggatcctcgcagacaagggctaaagtgctcg

SEQ ID NO: 34 - DNA, 68 bp, synthetic construct
  source 1..68: mol_type = other DNA
ggtaagccctacgagggggagcagacggtatgagacgggatcccgttcacctgacagcaaacgtgcgt